(19) United States
(12) Patent Application Publication
(10) Pub. No.: US 2021/0158957 A1
(43) Pub. Date: May 27, 2021

(54) SYSTEMS AND METHODS FOR MONITORING THE NEEDS OF A PERSON AND SELECTING A SERVICE PROVIDER

(71) Applicant: MASTERCARD INTERNATIONAL INCORPORATED, Purchase, NY (US)

(72) Inventors: Shawn Mehrhoff, Saint Ann, MO (US); Christopher T. Scholl, Saint Peter's, MO (US); James W. Barkhurst, Fenton, MO (US)

(21) Appl. No.: 16/696,217

(22) Filed: Nov. 26, 2019

(51) Int. Cl. G16H 40/67; G16H 15/00; G16H 10/60; G06Q 40/00

(52) U.S. Cl. CPC G16H 40/67; G16H 15/00; G16H 80/00; G06Q 40/12; G16H 10/60

(57) ABSTRACT

A computer-implemented method for aiding a caregiver in monitoring the needs of a person includes monitoring a plurality of transaction parameters of the person and determining if at least one of the plurality of transaction parameters being monitored satisfies a transaction parameter alert criterion. In response to at least one of the transaction parameters satisfying the transaction parameter alert criterion, a first need of the person is determined. Further, a service provider database is queried to retrieve a first service provider record having a service provider description associated with the first need of the person. A referral message identifying the service provider associated with the first service provider record is transmitted to the caregiver.

### BACKGROUND

The field of the present disclosure relates generally to monitoring the needs of a person, and more particularly to systems and methods for identifying a service provider to address the identified needs of the person.

In some known cases, a person may experience difficulty performing or completing various activities associated with daily living, such as shopping for groceries, filling prescriptions, scheduling and attending appointments, maintaining a residence (e.g., lawn care, home repairs, etc.), and/or selecting and hiring a service provider (i.e., a plumber, a home repair service person, an electrician, etc.). The person may suffer from an illness or condition that impedes the ability of the person to complete these activities, for example, the person may have limited mobility, and/or memory disorders, etc. The person may wish to hire a service provider to help complete activities; however, the person may be apprehensive about choosing a service provider. The person may wish to hire the best service provider, i.e., least expensive, most reliable, most experienced, etc. It may be especially difficult to select the best service provider if there are multiple service providers to choose from. In some cases, the person may wish to hire a service provider they previously hired but is unable to remember which service provider it was.

In some cases, a caregiver may be responsible for monitoring the needs of the person and/or helping the person complete activities. In some cases, the caregiver may be unable to visit the person on a frequent or regular basis. For example, the caregiver may have prior time commitments, such as full or part-time employment. In other cases, the caregiver may live a substantial distance away from the person making it time consuming for the caregiver to visit the person. In these types of cases, it may be difficult for the caregiver to confirm and/or monitor the needs of the person and to assess whether the person is completing specific tasks. For example, the caregiver may be unable to confirm that the person is paying bills on time, attending doctors' appointments, and/or taking prescribed medications. As such, the caregiver may attempt to monitor the needs of the person by periodically checking in with the person, i.e., calling or texting, to confirm that the person has completed specific tasks and/or to monitor the needs of the person. In some cases, the person may be hesitant and/or resistant to conveying their needs to the caregiver. Additionally, a caregiver may wish to aid the person in selecting and hiring a service provider to address a need of the person, but may also be apprehensive in selecting the best service provider from a list of service providers.

It may be advantageous to have methods and systems which assist the caregiver in monitoring the needs of the person and assist the caregiver and/or the person in selecting a service provider which may be hired to address the needs of the person.

### BRIEF DESCRIPTION

In one aspect, a computer system for aiding a caregiver in monitoring the needs of a person is provided. The computer system includes at least one processor in communication with a transaction database and a service provider database. The transaction database stores a plurality of transactions records associated with a plurality of transactions each initiated by one of a plurality of account holders at one of a plurality of merchants. Each transaction record includes a merchant identifier associated with the respective merchant and an account identifier associated with the respective account holder, and at least one of the account holders is the person. The service provider database stores a plurality of service provider records each associated with one of a plurality of service providers. Each service provider record includes a service provider identifier, a service provider contact data, and a service provider description associated with the types of goods and/or service provided by the service provider. The service provider description is associated with a need of the person. The at least one processor is configured to monitor a plurality of transaction parameters of the person, including monitoring the transaction database for transaction records including the account identifier associated with the person and determine if at least one of the plurality of transaction parameters being monitored satisfies a transaction parameter alert criterion. In response to at least one of the transaction parameters satisfying the transaction parameter alert criterion, the at least one processor determines a first need of the person. The at least one processor then queries the service provider database to retrieve a first service provider record having the service provider description associated with the first need of the person. The at least on processor also transmits a referral message to the caregiver. The referral message including the first need of the person and identifies the service provider associated with the first service provider record.

In another aspect, a computer-implemented method for aiding a caregiver in monitoring the needs of a person is provided. The method implemented using a computing device including a processor in communication with a memory device for storing data The method includes a transaction database storing a plurality of transactions records associated with a plurality of transactions each initiated by one of a plurality of account holders at one of a plurality of merchants. Each transaction record includes a merchant identifier associated with the respective merchant and an account identifier associated with the respective account holder. At least one of the account holders is the person. The method also includes a service provider database storing a plurality of service provider records each associated with one of a plurality of service providers. Each service provider record includes a service provider identifier, a service provider contact data, and a service provider description associated with the types of goods and/or service provided by the service provider. The service provider description is associated with a need of the person. The at least one processor is in communication with the transaction database and the service provider database. The at least one processor is configured to monitoring a plurality of transaction parameters of the person, including monitoring the transaction database for transaction records including the account identifier associated with the person and determining if at least one of the plurality of transaction parameters being monitored satisfies a transaction parameter alert criterion. In response to at least one of the transaction parameters satisfying the transaction parameter alert criterion, the at least one processor determines a first need of the person. The at least one processor also queries the service provider database to retrieve a first service provider record having the service provider description associated with the first need of the person. The at least one processor also transmits a referral message to the caregiver. The referral message includes the first need of the person and identifies the service provider associated with the first service provider record.

In a further aspect, a non-transitory computer-readable storage medium that includes computer-executable instructions for aiding a caregiver in monitoring the needs of a person is provided. The non-transitory computer-readable storage medium includes a transaction database, a service provider database, and at least one processor in communication with the transaction database and the service provider database. The transaction database stores a plurality of transactions records associated with a plurality of transactions each initiated by one of a plurality of account holders at one of a plurality of merchants. Each transaction record includes a merchant identifier associated with the respective merchant and an account identifier associated with the respective account holder. At least one of the account holders is the person. The service provider database storing a plurality of service provider records each associated with one of a plurality of service providers. Each service provider record includes a service provider identifier, a service provider contact data, and a service provider description associated with the types of goods and/or service provided by the service provider. The service provider description is associated with a need of the person. The at least one processor is configured to monitor a plurality of transaction parameters of the person, including monitoring the transaction database for transaction records including the account identifier associated with the person and determining if at least one of the plurality of transaction parameters being monitored satisfies a transaction parameter alert criterion. In response to at least one of the transaction parameters satisfying the transaction parameter alert criterion, the at least one processor determines a first need of the person. The at least one processor queries the service provider database to retrieve a first service provider record having the service provider description associated with the first need of the person. Also, the at least one processor transmits a referral message to the caregiver. The referral message includes the first need of the person and identifies the service provider associated with the first service provider record.

### BRIEF DESCRIPTION OF THE DRAWINGS

FIGS. 1-4 show example embodiments of the methods and systems described herein.

FIG. 1 is a simplified schematic diagram of an example process flow between components of a caregiving system including a caregiving computing device for monitoring the needs of a person and/or identifying a service provider that may be hired by the person to assist with the need.

FIG. 2 is a process flow diagram of an example of a caregiving process for monitoring the needs of a person and identifying a service provider.

FIG. 3 illustrates an example configuration of the caregiving computing device shown in FIG.1.

FIG.4 illustrates an example configuration of a person computing device shown in FIG. 1.

### DETAILED DESCRIPTION

The systems and methods described herein are directed to assisting a caregiver in monitoring and addressing the needs of a person, and more specifically, monitoring a plurality of parameters of the person, determining a need of the person, and identifying a service provider that may be hired to address the needs of the person. In some cases, the person (e.g., a person with impairments related to old age, disability, and/or disorders, etc.) may rely on the caregiver (e.g., family member, caretaker, nurse, guard, etc.) for assistance in completing various tasks associated with activities of daily living. In some cases, the caregiver may have a substantial role in the care of the person and as such, the caregiver may monitor one or more parameters associated with the person on a frequent or daily basis in order to determine the needs of the person. In other cases, the person may need limited assistance; however, the person and/or the caregiver may wish to have the peace of mind confirming that the needs of the person are being addressed.

The system and processes described herein include transmitting and receiving a plurality of messages between the person, the caregiver, a caregiving system, and a payment processing network. The caregiving system may be communicatively connected to a transaction database, a service provider database, and/or a personal care database. The transaction database includes a set of transaction records each associated with a corresponding one of a plurality of transactions by one of a plurality of account holders with one of a plurality of merchants. Each transaction record includes a transaction identifier (ID) associated with the respective transaction, an account number associated with the respective account holder, and a merchant identifier (ID) associated with the respective merchant. Each of the transaction records may also include transaction data including a date/time of the transaction, a transaction amount, and/or a description of the goods or services purchased in the transaction. In other words, the transaction database includes information regarding historical payment transactions between the plurality of account holders and the plurality of merchants. The payment processing network may build the transaction database by extracting data from payment transaction messages routed over the payment processing network, building transaction records from the extracted data, and storing the transaction records to the transaction database.

The caregiving system may be associated with a caregiving computing device. The caregiving computing device may retrieve at least a subset of transaction records from the transaction database. The caregiving computing device may determine a service provider that meets a need of the person from a set of merchants based on data contained in the set of transaction records. The service provider may be selected based on predefined metrics, e.g., a proximity of the merchant to the person, the most commonly used merchant, the cheapest merchant, and/or the merchant with the least amount of “negative” transaction (e.g., chargebacks). In some embodiments, the caregiver computing device may sort the transaction records by the type of merchant in order to categorize the merchants based on the types of goods and/or service provided or sold by the merchant. For example, a set of merchants may be categorized into, for example and without limitation, a plumbing service category, a landscaping category, a home repair category, etc. Each merchant category may include one or more merchants. The caregiving computing device may determine a scoring parameter for one or more predefined metrics, for each merchant contained in each merchant category, based on transaction data contained in the transaction records. The scoring parameter may be used to select a service provider from a plurality of merchants within a merchant category. The scoring parameter may be based on, for example and without limitation, the proximity of the merchant, the number of transactions occurring with the merchant, a number of negative transactions (i.e., refunds, chargebacks), the average amount per transaction, and/or any combination of these. In other words, the caregiver computing device may use information contained in the transaction database to determine, from a set of merchants, service providers that best satisfy the one or more predefined metrics.

The caregiving system may build the service provider database by storing a plurality of service provider records to the service provider database. Specifically, the service provider database includes a set of service provider records, with each record corresponding to one of the plurality of merchants. Each service provider record includes a merchant description and service provider contact information. The merchant description includes a description of the goods and/or service provided or sold by the service provider.

In some embodiments, one or both of the caregiver and the person may enroll (e.g., register) with the caregiving system. During registration, the person and/or the caregiver may provide personal details associated with the person to the caregiving system. Personal details may include, for example and without limitation, the person's age, weight, medical conditions, medications, and/or personal calendar, etc. The registration process may also capture details about the caregiver, such as the caregiver's calendar. In some embodiments, registration includes an authorization for the caregiving system to track or monitor a plurality of parameters associated with the person. In this example embodiment, registration may authorize the caregiving system to track and monitor payment transactions initiated by the person. The caregiving computing device may generate a personal care record for the person based on data received during registration or subsequent updates submitted by the person or the caregiver. The caregiving computing device may build the personal care database by storing a plurality of personal care records to the personal care database.

In some embodiments, the caregiving system may continuously or periodically monitor (i.e., by sampling, measuring, evaluating) parameters (e.g., a tracking parameter and/or a transaction parameter) associated with the needs and/or activities of the person. The caregiving system may continuously or periodically monitor a transaction parameter associated with payment transactions initiated by the person. The person may initiate a purchase transaction (“payment transaction”) by providing payment credentials (e.g., a credit or debit card number, a bank account number, user login information corresponding to saved payment credentials, digital wallet information, etc.) to a merchant for the exchange of goods and/or services. After the person initiates a purchase transaction, the payment processing network typically stores a transaction record in the transaction database as discussed above. In some examples, the payment processing network may identify, during processing of the payment transaction, the payment credentials as belonging to the person registered with the caregiving system and separately transmit the transaction record to the caregiving computing device. Alternatively, the caregiving computing device may monitor (e.g., periodically query) the transaction database for new transaction records associated with the person. In response to receiving or retrieving the personal transaction record, the caregiving system may transmit a notification message to the caregiver indicating that the person has initiated a payment transaction. In some example embodiments, the caregiving system may determine if a transaction parameter contained in the personal transaction record satisfies a transaction parameter alert criterion. For example, the caregiver may wish to confirm that the person has attended an appointment, such as a doctor's appointment, with a health care service provider. While attending the appointment, the person may initiate a payment transaction, for example a copayment, with the healthcare service provider. The transaction record associated with the person's payment credentials and the healthcare service provider may trigger the caregiving system to transmit a notification message to the caregiver indicating that the person has attended the appointment.

In some embodiments, the caregiving computing device may continuously or periodically monitor a tracking parameter associated with the person. The caregiving system may receive and/or retrieve a plurality of tracking messages, including the tracking parameter, from one or more devices associated with the person. Devices associated with the person may include, for example and without limitation, a mobile device, a computing device, a medical device, and/or a biometric device. For example, the caregiving system may be linked to a wearable biometric device, e.g., Fitbit, step counting device, and/or heart rate monitor. Additionally or alternatively, the caregiving system may be linked to a medical device of the person, for example and without limitation, a pacemaker, a heart monitor, and/or a glucose monitoring system. The tracking parameter associated with the person may include, for example and without limitation, a biometric parameter of the person, such as weight, heart rate, steps taken, blood sugar levels, etc. Additionally or alternatively, the tracking parameter may include Global Positioning System (GPS) data that may be used to determine the location of the person.

In certain embodiments, the caregiving computing device determines if the tracking parameter satisfies a tracking parameter alert criterion. If the tracking parameter satisfies the tracking parameter alert criterion, the caregiving computing device may determine a need of the person and/or transmit a notification message to the caregiver. For example, the caregiving computing device may receive a plurality of tracking messages from a glucose monitoring device of the person. The tracking messages including the tracking parameter of blood sugar levels associated with the person. The caregiving computing device may set a tracking parameter alert criterion to include a blood sugar level varying outside an accepted range. As such, if the blood sugar satisfies the tracking parameter alert criterion, then the caregiving computing device transmits a notification message to the caregiver indicating the blood sugar level of the person.

In some embodiments, either one or both the person and/or the caregiver are communicatively coupled to the caregiving system, such that the person and/or the caregiver may transmit and/or receive a plurality of messages with the caregiving system. For example, either one or both of the caregiver and/or the person may transmit a query message and/or a request message to the caregiving system. The query message may be associated with asking for a recommendation for a service provider. For example, the person may need assistance cleaning the gutters of their residence. The person and/or the caregiver may transmit a query message to the caregiving computing device, requesting a recommendation for a gutter cleaning service provider.

The caregiving computing device may then retrieve a service provider record from the service provider database, for example by applying one or more predefined metrics as discussed above, for a service provider associated with a gutter cleaning service. The request message may be associated with the tracking or monitoring of a tracking parameter and/or a transaction parameter of the person. For example, the caregiver may transmit a request message to the caregiving system requesting to be notified if the person initiates a purchase transaction with specific merchants. For example, the caregiver may wish to know when the person is traveling and may request to be notified when a payment transaction is initiated with a transportation service provider. In other words, one of the caregiver and/or the person may request to set the transaction parameter alert criterion and/or the tracking parameter alert criterion.

At least one technical problem to be solved by the systems and methods provided herein includes: (i) ability of a remote caregiver to objectively determine a well-being of a person, (ii) ability of a remote caregiver to identify needs of a person, (iii) ability to timely notify the caregiver of the need of the person, and (iv) ability to remotely select a quality service provider for the person.

The technical effect of the systems and processes described herein may achieved by performing at least one of the following steps: (i) monitoring a plurality of transaction records to identify personal transaction parameters of the person, (ii) determining if at least one of the transaction parameters being monitored satisfies a transaction parameter alert criterion, (iii) monitoring a tracking parameter of the person from one or more devices associated with the person, (iv) determining if at least one of the tracking parameters being monitored satisfies a tracking parameter alert criterion, (v) in response to at least one of the transaction parameters or tracking parameters satisfying the transaction or tracking parameter alert criterion, determining a first need of the person, (vi) querying the service provider database to retrieve a first service provider record having the service provider description associated with the first need of the person, and (vii) transmitting a referral message to the caregiver, the referral message including the first need of the person and identifying the service provider associated with the first service provider record.

A technical effect or improvement provided by the systems and processes described herein include at least one of: (i) enabling a remote caregiver to objectively monitor a well-being of a person, including monitoring transaction records initiated by the person, (ii) facilitating the caregiver in determining the needs of the person, (iii) enabling a remote caregiver to objectively identify an appropriate service provider from a list of merchants, and (iv) recommending a service provider that may be hired by the caregiver and/or the person to address a need of the person.

As used herein, a processor may include any programmable system including systems using micro-controllers, reduced instruction set circuits (RISC), application specific integrated circuits (ASICs), logic circuits, and any other circuits or processor capable of executing the functions described herein.

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and thus are not limiting as to the types of memory usable for saving of a computer.

In one embodiment, a computer program is provided, and the program is embodied on a computer readable medium. In an example embodiment, the data optimization system is executed on a single computer system, without requiring a connection to a server computer. In a further embodiment, the system is being run in a Windows® environment (Windows is a registered trademark of Microsoft Corporation, Redmond, Wash.). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

As used herein, the terms “transaction card,” “financial transaction card,” and “payment card” refer to any suitable transaction card, such as a credit card, a debit card, a prepaid card, a charge card, a membership card, a promotional card, a frequent flyer card, an identification card, a prepaid card, a gift card, a card that is part of a digital wallet, and/or any other device that may hold payment account information, such as mobile phones, smartphones, personal digital assistants (PDAs), key fobs, and/or computers. Each type of transaction card can be used as a method of payment for performing a transaction. As used herein, the term “payment account” is used generally to refer to the underlying account associated with the transaction card.

The following detailed description illustrates embodiments of the disclosure by way of example and not by way of limitation. It is contemplated that the disclosure has general application to processing financial transaction data by a third party in industrial, commercial, and residential applications.

As used herein, an element or step recited in the singular and proceeded with the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

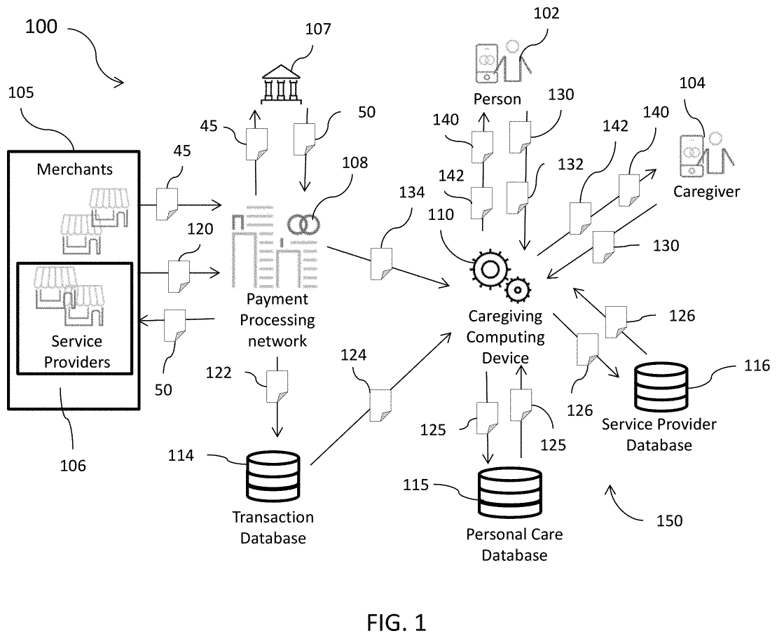

FIG. 1 is a schematic diagram of an example process flow 100 using a caregiving system 150 for monitoring a plurality of parameters of a person 102, determining a need of person 102, and identifying a service provider 106 to meet the determined need in accordance with the present disclosure. In this example, process flow 100 includes an exchange of a plurality of messages between person 102 (e.g., a dependent and/or a patient), a caregiver 104, a plurality of merchants 105, a payment processing network 108, and a caregiving computing device 110 associated with caregiving system 150. In some example embodiments, caregiving computing device 110 may be integrated with payment processing network 108. In other embodiments, caregiving computing device 110 may be separate from, but communicatively connected to a transaction database 114 associated with and/or otherwise configured to receive transaction records 122 from, payment processing network 108.

Payment processing network 108 includes a set of proprietary communications standards for the exchange of financial transaction data and the settlement of funds between financial institutions. Payment processing network 108 is configured to process payment/purchase transactions for a plurality of cardholders (such as person 102) with plurality of merchants 105, by transmitting various authorization request and response messages between parties to the transaction (e.g., the respective merchant 105, an acquirer bank (not shown) associated with the merchant 105, and an issuer 107 of a payment card of the respective cardholder). As used herein, “payment processing network” refers broadly to the network and/or to one or more computing devices associated therewith (e.g., payment processors or payment processing computing devices). Payment processing network 108 may store a merchant description for each merchant 105. The merchant description includes descriptions of the types of good and/or service provided or sold by merchants 105. In some cases, merchants 105 may register with caregiving system 150 by transmitting a description of merchant 105 in a merchant message 120 to caregiving system 150. In some examples, merchants 105 may not be required to register with caregiving system 150 and/or caregiving system 150 may determine the merchant description using other methods. For example, payment processing network 108 may determine the type of goods and service sold or provider by merchants 105 using a merchant category code previously assigned to merchant 105, and this information may be provided to caregiving system 150. The merchant category code is used to distinguish merchants 105 by the type of goods and/or service sold and/or provided by merchants 105.

Upon initiation of a purchase transaction, e.g. by person 102, merchant 105 submits an authorization request message 45 for the transaction through payment processing network 108 to issuer 107 (also known as an issuer bank) for transaction authorization. Issuer 107 processes the request message 45, e.g. by determining whether the cardholder's account is in good standing and has available credit to cover the transaction amount, and transmits an authorization response message 50 back through the payment processing network 108 to merchant 106. Authorization response message 50 indicates either denial or approval of the purchase transaction.

In the example, payment processing network 108 is communicatively coupled to transaction database 114 such that the payment processing network 108 may transmit and/or store a transaction record 122 to transaction database 114 in order to create and/or build transaction database 114. In other words, transaction database 114 contains set of transaction records 122. Each transaction record 122 is associated with a corresponding payment/purchase transaction by one of a plurality of cardholders with one of merchants 105. More specifically, each transaction record 122 includes a transaction identifier (ID) associated with the respective transaction, an account number associated with the respective account holder, and a merchant identifier (ID) associated with merchant 105. Each of transaction record 122 may also include transaction data, for example and without limitation, a date/time of the transaction, a transaction amount, and/or a description of the goods or services purchased and/or provided in the transaction. In other words, transaction database 114 includes information regarding payment transactions occurring with a plurality of merchants 105 with a plurality of account holders. At least one of the account holders is the person 102. For merchants registered with caregiving system 150, transaction record 122 may further include the merchant description 120. Alternatively, caregiving system 150 may match transaction record 122 with the corresponding merchant description 120 based on the merchant ID.

Caregiving computing device 110 is communicatively coupled to transaction database 114. In some examples, payment processing network 108 provides caregiving computing device 110 access to transaction database 114 maintained by payment processing network 108. Alternatively, payment processing network 108 transmits transaction records 122 to caregiving computing device 110, and caregiving computing device 110 adds transaction records 122 to transaction database 114 maintained by caregiving computing device 110. Accordingly, caregiving computing device 110 may receive and/or retrieve a subset of transaction records 124 from transaction database 114. Subset of transaction records 124 may be selected and/or filtered from transaction records 122 stored in transaction database 114 using a filtering criterion. The filtering criterion may be determined from the merchant description associated with transaction record 122. In other words, caregiving computing device 110 may retrieve/receive subset of transaction records 124 from transaction database 114 based on a parameter associated with merchant 105, in order to limit or restrict the number of transaction records 122 being received and/or retrieved from transaction database 114. For example, the filtering criteria may be based on the geographical location of merchant 105. For example, subset of transaction records 124 may exclusively include merchants 105 having a location within a 30 mile radius from a residential location associated with person 102 in personal care database 115. Additionally or alternatively, the filtering criterion may be based on the types of goods and services provided or sold by merchants 105. For example, subset of transaction records 124 may exclusively include merchants 105 that perform lawn care services, i.e., landscaping service providers, lawn mowing services etc. In other examples, the subset of transaction records 124 may exclusively include merchants 105 that perform plumbing services.

Caregiving computing device 110 analyzes subset of transaction records 124 in order to identify a service provider 106 from among merchants 105 included within subset of transaction records 124. Service provider 106 may be selected by applying predefined metrics to merchants 105 included within the subset of transaction records 124. In some embodiments, service provider 106 may be associated with the most commonly hired merchant, the least expensive merchant, and/or the merchant with the least amount of chargebacks. For example, the predefined metrics may include one or more scoring parameters. The scoring parameter may be based on, at least in part, data contained in subset of transaction records 124. In other words, a scoring parameter will be determined for each merchant 105 contained within subset of transaction records 124.

For example, subset of transaction records 124 may be filtered to include a set of merchants 105 associated with a landscaping or lawn care service. The predefined metrics selected by the person 102 and/or the caregiver 104 may be a landscaping service provider which charges the least amount for a mowing service. As such, the scoring parameter may be based on the average transaction amount per transaction occurring with the set of merchants 105 associated with a landscaping or lawn care service. Additionally or alternatively, the predefined metrics selected by the person 102 and/or the caregiver 104 may be a landscaping service provider that is the most commonly used for a mowing service. As such, the scoring parameter may be based on a total number of transactions occurring with the set of merchants 105 associated with a landscaping or lawn care service, over a period of time. In other examples, the predefined metrics selected by the person 102 and/or the caregiver 104 may be whether the service provider 106 was previously hired by person 102. As such, the scoring parameter may be based on transactions occurring with the set of merchants 105 associated with a landscaping or lawn care service that also include the payment credentials of the person 102. In other words, the scoring parameter may be used to determine if person 102 had previously hired merchant 105. Additionally or alternatively, the predefined metrics and/or the scoring parameter may include a combination of determined parameters. For example, the predefined metrics may be to select five merchants 105 having the five lowest amounts charged based on subset of transaction records 124, and then to select the most commonly used merchant 105 from among the five as the service provider 106.

In response to caregiving computing device 110 identifying service provider 106, caregiving computing device 110 may transmit/store a service provider record 126 in service provider database 116. Service provider record 126 includes data associated with service provider 106 to facilitate identification of a previously selected service provider 126 when the corresponding need of the person 102 arises again. In some examples, service provider record 126 includes a look-up index. The look-up index may be associated with the types of goods and/or services provided or sold by service provider 106. Caregiving computing device may use the look-up index to look-up (i.e., retrieve) service provider record 126 for service provider 106 in response to a subsequent need of person 102.

In other words, caregiving computing device 110 determines a scoring parameter for each merchant 105 contained in the subset of transaction records 124 retrieved from the transaction database 114. The scoring parameter may be used to identify service provider 106 from the set of merchants 105. Each of service providers 106 is associated with one or more of the needs of the person. For example, service provider database 116 contains a plurality of service provider records 126, wherein each service provider record 126 includes a merchant description that can be used to determine the types of goods and/or services that the corresponding service provider 106 sells or provides. The look-up index may be used to retrieve service provider 106 from service provider database 116 based on the need of person 102.

Person 102 and/or caregiver 104 may register with caregiving system 150 during a registration process by transmitting one or more registration messages 130 to the caregiving computing device 110. In other embodiments, person 102 and caregiver 104 may transmit registration message 130 to payment processing network 108 in order to register with caregiving system 150. Registration message 130 may include a set of information associated with person 102 and/or caregiver 104. The set of information may include parameters specific to person 102, such as age, weight, location of residency, personal calendar (i.e., appointments, medication schedules, etc.), medical information including dietary restrictions, medications, allergies, etc. The set of information may include alternative or additional parameters associated with person 102. Further, in some examples, registration message 130 includes authorization to track a set of parameters of person 102. For example, registration message 130 may authorize the caregiving system 150 to track the payment transactions initiated by person 102, i.e., authorize access by caregiving system 150 to transaction records 122 including an account number associated with person 102 (i.e., personal transaction records 134).

In some embodiments, caregiving computing device 110 is communicatively coupled to a personal care database 115. The personal care database may store a plurality of personal care records 125. Each personal care record 125 includes a receiver identifier associated with one of a plurality of care receivers (such as person 102), an item identifier associated with one of a plurality of goods or services required by the respective care receiver, and a schedule data identifying a schedule on which the respective good or service is required. Caregiving computing device 110 may generate personal care records 125 based on data contained in registration messages 130. Additionally or alternatively, caregiving computing device 110 may generate personal care records 125 using data obtained from other sources, e.g., medial charts transmitted from a healthcare service provider. The caregiving computing device 110 may retrieve one or more personal care records 125 from personal care database 115. In some embodiments, personal care database 115 may be filtered based on a filtering criterion in order to decrease or limit the total number of personal care records 125 that will be considered in a given database operation.

In some embodiments, caregiving computing device 110 is communicatively coupled to one or more devices associated with person 102 and/or caregiver 104. As such, caregiving computer device 110 may receive and/or retrieve a plurality of a tracking message 132 from the one or more devices associated with person 102 and/or caregiver 104. The devices may include a mobile computing device, such as a computer, a laptop, and/or a mobile phone associated with person 102 and/or caregiver 104. In other examples, caregiving computing device 110 may be connected to other devices associated with person 102. For example, caregiving computing device 110 may be communicatively connected to, for example and without limitation, a wearable biometric tracking device (e.g., Fitbit, fitness watch, step counter, scale, etc.) and/or a medical device or implant (e.g., pacemaker, heartrate monitor, blood pressure machine, and/or wearable glucose monitoring system). Tracking message 132 includes a set of tracking parameters associated with the person 102. For example, caregiving computing device 110 may track a set of tracking parameters associated with a biometric of person 102 based on data received/retrieved from the wearable biometric tracking devices. For another example, caregiving computing device 110 may retrieve tracking message 132 from a mobile computing device associated with person 102, wherein tracking message 132 includes a tracking parameter associated with the geographical location of the mobile computing device.

In certain embodiments, caregiving computing device 110 also receives and/or retrieves a personal transaction record 134 from payment processing network 108. Personal transaction record 134 includes a transaction parameter associated with a payment transaction initiated by person 102. Personal transaction record 134 may be transmitted to caregiving computing device 110, in real-time or near real-time, i.e., personal transaction record 134 may be transmitted to caregiving computing device 110 by payment processing network 108 at the same or approximately the same time as when person 102 initiated the payment transaction. In other examples, receipt or retrieval of personal transaction record 134 may be delayed from the initiation of a payment transaction such that personal transaction records 134 may be retrieved periodically in a batch, e.g., via a query submitted by caregiving computing device 110 to transaction database 114.

Caregiving computing device 110 may receive a plurality of tracking messages 132 from person 102 and/or a plurality of personal transaction records 134 from payment processing network 108. In some examples, caregiving computing device 110 may receive/retrieve tracking message 132 and personal transaction record 134 regularly or with a predetermined frequency. In other examples, caregiving computing device 110 may receive and/or retrieve tracking messages 132 and/or personal transaction records 134 using a trigger criterion. For example, the trigger criterion may be every instance a payment transaction is initiated by person 102. In other words, caregiving computing device 110 may receive personal transaction record 134 each time a payment transaction is initiated by person 102. Additionally or alternatively, the trigger criterion may be each instance a payment transaction is initiated by person 102 with a specific merchant 105. In other words, personal transaction record 134 may be transmitted only when a payment transaction is initiated with specific service providers 106. For example, the trigger criterion includes payment transactions initiated with a health care service provider 106, such that each time person 102 initiates a purchase transaction with the healthcare service provider, a personal transaction record 134 will be transmitted to caregiving computing device 110.

Tracking messages 132 and personal transaction records 134 include a set of parameters (tracking parameters and transaction parameters, respectively) being monitored or tracked by caregiving computing device 110. In other words, caregiving computing device 110 monitors the tracking parameters and/or the transaction parameters by sampling, saving, and/or calculating data contained in tracking messages 132 and personal transaction records 134. For example, caregiving computing device 110 may use the tracking parameters contained in the tracking message 132 to monitor the geographical location of person 102, i.e., caregiving computing device 110 may periodically monitor the location of person 102 from data obtained from Global Positioning data contained in tracking message 132. In other examples, caregiving computing device 110 may monitor the number and/or timing of payment transactions occurring with a healthcare provider, i.e., caregiving computing device 110 may count the number of personal transaction records 134 for payment transactions initiated with a healthcare service provider. Additionally or alternatively, caregiving computing device 110 may determine and/or monitor any other suitable parameters that enable the caregiving computing device 110 to function as described herein.

In some embodiments, caregiving computing device 110 may determine if the monitored transaction parameters and/or tracking parameters satisfy a criterion, more particularly a transaction alert criterion or a tracking alert criterion, respectively. Caregiving computing device 110 may be configured to determine a need of person 102 in response to a monitored transaction parameter and/or tracking parameter satisfying the respective transaction alert criterion and/or the tracking alert criterion. For example, the transaction parameter may be an amount per transaction and the transaction alert criterion may be an upper limit amount. In other words, caregiving computing device 110 may monitor amounts of individual transactions initiated by person 102 to determine if the amount per transaction exceeds the upper limit. If person 102 initiates a purchase transaction exceeding the upper limit, thereby satisfying the transaction alert criterion, then caregiving computing device 110 may determine a need of the person. In some cases, the tracking parameter alert criterion and the transaction parameter alert criterion may be used together to determine a need of the person. In response to caregiving system 150 determining the needs of person 102, caregiving computing device 110 may transmit a notification message 140 to at least one of caregiver 104 and person 102. Notification message 140 includes information that may be used to indicate to person 102 and/or caregiver 104 the need of the person.

To further illustrate the transaction alert criterion and the tracking alert criterion, an example is provided. Caregiving computing device 110 may monitor a tracking parameter including scheduling data associated with person 102. The scheduling data may be obtained from, for example, personal care record 125 contained in the personal care database 115. The scheduling data may be used to determine that person 102 has a medical appointment with a healthcare service provider for a specific data and time. In this first example, monitoring the transaction parameters may include monitoring the transactions of person 102 to detect transactions between person 102 and the merchant ID associated with the healthcare provider. In addition, the transaction parameter alert criterion may include confirming that a payment transaction was initiated by person 102 with the healthcare service provider on the specific date for when the medical appointment is scheduled. In other words, if person 102 attends the appointment and pays a copayment, then this may serve as an indication that person 102 has attended their appointment. If no payment transaction is detected between person 102 and the healthcare service provider on the specific date, the transaction parameter alert criterion is satisfied and caregiving system 150 presumes that the person 102 has failed to attend the appointment. In response, caregiving computing device 110 may determine of the need of person 102 includes rescheduling their appointment. Caregiving computing device 110 may also transmit notification message 140 to the caregiver, indicating that person 102 may not have attended their appointment and potentially will need to reschedule.

The quantity and types of monitored tracking parameters and/or transaction parameters may be determined by caregiving system 150. Likewise, the tracking parameter alert criterion and the tracking alert criterion may be determined by the caregiving system 150. Additionally and/or alternatively, person 102 and/or caregiver 104 may select and/or request the quantity and types of monitored tracking parameters, transaction parameters, and the associated tracking parameter alert criterion and/or transaction parameter alert criterion. To further illustrate this, another example is provided. During the registration process, caregiver 104 may request that caregiving computer system 110 monitor whether person 102 makes a significant purchase, e.g., person 102 spends an amount over $500. As such, caregiving computing device 110 may monitor the transaction parameters including the transaction amount for each personal transaction record 134. Caregiving computing device 110 may set the transaction parameter alert criterion including payment transactions exceeding $500.00, such that when person 102 initiates a payment transaction exceeding $500.00, caregiving computing device 110 may transmit notification message 140 to caregiver 104 indicating that person 102 has made a significant purchase. In other words, in the event that person 102 makes a purchase transaction of an amount exceeding $500.00, for example $501.00, then caregiving computing device 110 transmits notification message 140 to caregiver 104. Additionally or alternatively, notification message 140 may include additional details associated with data contained in the personal transaction record 134, for example and without limitation, information about merchant 105 at which the purchase transaction is taking place.

Another example is provided to further illustrate the functionality of caregiving system 150. In some embodiments, caregiver 104 may wish to confirm that person 102 has paid a monthly bill, for example and without limitation, rent, utilities, and the like. As such, caregiving computing system 110 may track a recurring payment transaction, i.e., a payment transaction occurring each month with a specific service provider 106 for a specific amount and/or a specific range of amounts. For example, caregiver 104 may wish to confirm that person 102 has paid the utility bill each month within a range of values of $75.00 to $200.00. Caregiver 104 may request to be notified if the payment amount of the monthly utility bill exceeds the upper range of values, i.e., payment transaction initiated with a utility service provider exceeds $200.00. Caregiving computing system 110 may monitor the personal transaction records 134 of the person 102 for transactions having the transaction parameter of merchant ID matching the utility service provider. Caregiving computing device 110 may set the transaction alert criterion to include personal payment record 134 with the utility service provider that exceeds $200.00. In other words, if person 102 initiates a payment transaction which exceeds $200.00 with the utility service provider, then caregiving computing device 110 may transmit notification message 140 to caregiver 104 indicating that person 102 is initiating a purchase transaction exceeding $200.00 with the utility service provider.

In certain embodiments, caregiving computing device 110 may determine a need associated with person 102. Caregiving computing device 110 may perform a look-up operation to retrieve service provider record 126 stored in service provider database 116 based on the need determined by caregiving computing device 110. Caregiving computing device may then transmit a recommendation message 142 to at least one of person 102 and/or caregiver 104. After the need of person 102 has been determined, then caregiving computing device 110 may identify service provider 106 that may be hired to complete a task associated with the need of the person.

For example, person 102 and/or caregiver 104 may periodically hire a landscaping company to groom/mow the lawn of the residence associated with person 102. In some cases, person 102 may be neglectful and/or forgetful to hire the landscaping company to mow the lawn. For this example, the transaction parameter may be personal transaction records 134 having the merchant ID matching the landscaping service provider, and the transaction parameter alert criterion may include a threshold period of time over which no personal transaction records 134 are associated with the landscaping service provider. In other words, if person 102 neglects to hire the lawn care service provider to mow the lawn for an extended period of time, then caregiving computing device 110 may determine that the need of the person includes mowing the lawn of the residence of person 102. As such, caregiving computing device 110 performs a look-up operation to retrieve service provider record 126 associated with the landscaping service provider from service provider database 116. Caregiving computing device may transmit a referral message to person 102 and/or caregiver 104 indicating that the lawn at the residence of person 102 may need to be mowed and identifying the service provider 106 that may be hired to mow the lawn. In some examples, service provider database 116 may not include a record 126 for a landscape service, and person 102 and/or caregiver 104 may transmit a query message to the caregiving computing device 110. The query message may include a request for a recommendation for service provider 106 that may be hired to address a need of person 102, and caregiver computing device 110 may generate the recommendation as described above.

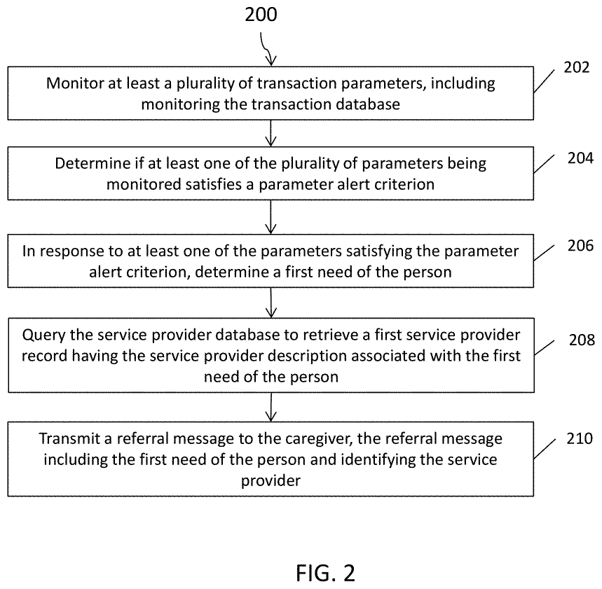

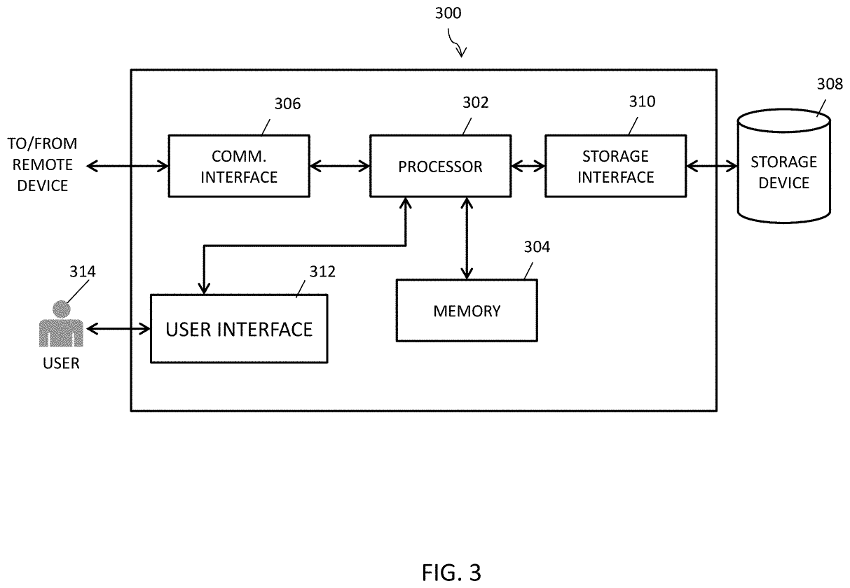

FIG. 2 is a process flow diagram of a method 200 for monitoring the needs of a person (e.g., person 102) and for identifying a service provider (e.g., service provider 106) that may be hired by at least one of the person or a caregiver (e.g., caregiver 104, and/or person 102). In the example embodiment, method 200 is implemented by a caregiving computing device (e.g., caregiving computing device 110). The caregiving computing device includes at least one processor 302 (shown in FIG. 3). The caregiving computing device is communicatively coupled to a transaction database (e.g., transaction database 114) and a service provider database (e.g., service provider database 116). In some embodiments, the caregiving computing device is also communicatively coupled to a personal care database (e.g., personal care database 115).

The transaction database stores a plurality of transaction records (e.g., transaction records 122) associated with a plurality of transactions each initiated by one of a plurality of account holders at one of a plurality of merchants (e.g., merchants 105), wherein each transaction record includes a merchant identifier associated with the respective merchant and an account identifier associated with the respective account holder, and wherein at least one of the account holders is the person.

The service provider database stores a plurality of service provider records (e.g., service provider records 126) each associated with one of a plurality of service providers, wherein each service provider record includes a service provider identifier, a service provider contact data, and a service provider description associated with the types of goods and/or service provided by the service provider, wherein the service provider description is associated with a need of the person. For example, the service provider identifier may be the merchant ID used by payment processing network 108, or another unique identifier assigned by caregiver computing device 110.

The personal care database stores a plurality of personal care records (e.g., personal care records 125), each personal care record including a receiver identifier associated with one of a plurality of care receivers, an item identifier associated with one of a plurality of goods or services required by the respective care receiver, and a schedule data identifying a schedule on which the respective good or service is required, wherein at least one of the plurality of care receivers is the person 102.

In some embodiments, at least one of the transaction database, the service provider database, and/or the personal care database may be filtered using a filtering criterion. Filtering the transaction database may be used to decrease or limit the total number of transaction records in the transaction database that will be considered in a given database operation. Likewise, filtering the service provider database may be used to decrease or limit the total number of service providers in the service provider database that will be considered in a given database operation. Further, filtering the personal care database may be used to decrease or limit the total number of personal care records that will be considered in a given database operation.

Method 200 includes the at least one processor monitoring 202 a plurality of transaction parameters of the person, as discussed above. Monitoring 202 the plurality of transaction parameters includes monitoring the transaction database for transaction records including the account identifier associated with the person. In some embodiments, a payment processing network (e.g., payment processing network 108) may transmit a personal transaction record (e.g., personal transaction record 134) associated with a payment transaction initiated by the person to the caregiving computing device, which stores the received records in transaction database 114 maintained by the caregiving computing device. Additionally or alternatively, the caregiving computing device has access to transaction database 114 associated with the payment processing network.

In some embodiments, the at least one processor may monitor a plurality of tracking parameters of the person, as discussed above. Monitoring the plurality of tracking parameter of the person may include the at least one processor retrieving a plurality of tracking messages (e.g., tracking messages 132) from a device associated with the person. The device associated with the person may include a personal computing device, for example and without limitation, a laptop, a mobile phone, a tablet, etc. In other example embodiments, the device associated with the person may include a medical device, for example and without limitation, a pacemaker, a heart rate monitor, a wearable biometric tracking device (i.e., set counter, a Fitbit, and the like) and/or a glucose monitoring system. The tracking messages obtained from the device includes a plurality of tracking parameters, for example and without limitation, the geographical location associated with the person, the heart-rate associated with the person, and/or a personal schedule (i.e., dates and times of appointments). In other words, the at least one processor may retrieve a plurality of tracking messages from one or more devices associated with the person to monitor a plurality of tracking parameters of the person. For example, method 200 may include monitoring the blood sugar levels of the person, as such the at least one processor may retrieve a tracking message from a glucose monitoring device of the person. In this example, the tracking messages may include the blood sugar level of the person. In yet another example, method 200 may include tracking the location of the person, as such, the at least one processor may retrieve a tracking message from the mobile device associated with the person. In this example, the tracking message may include the Global Positioning coordinates of the mobile device.

Method 200 further includes the at least one processor determining 204 if at least one of the plurality of transaction parameters being monitored satisfies a transaction parameter alert criterion. In response to the transaction parameter satisfying the transaction parameter alert criterion, the at least one processor determines 206 a need of the person. In other embodiments, method 200 further includes, the at least one processor determining if the tracking parameter satisfies a tracking parameter alert criterion. In response to the tracking parameter satisfying the tracking parameter alert criterion, the at least one processor determines a need of the person. For example, the transaction parameter may include recurring payments associated with rental payments of the person. The transaction parameter alert criterion may be associated with the person failing to pay the rental insurance, i.e., a missing recurring payment transaction with a property owner associated with the rental property. In this example, the at least one processor may determine that the need of the person is associated with initiating a payment transaction with the landlord of the rental property. In another example, the tracking parameter may be the location of the person, and the tracking alert criterion may be the location of the person exceeding a 100 mile radius from their residential location. In other words, if the person travels a distance greater than a 100 miles from their home, the tracking parameter satisfies the tracking alert criterion and as such, the need of the person may be associated with directions leading from the current location of the person to the location of the person's residence, and/or a transportation service that may transport the person to the residence.

Method 200 further includes querying 208 the service provider database to retrieve a first service provider record having the service provider description associated with the determined need of the person. In other words, after the at least one processor determines the need of the person then the at least one processor will retrieve a service provider from the service provider database that may be hired to aid the person in addressing the identified need. For example, if the need of the person is identified to be associated with a plumbing service, then the at least one processor will retrieve a service provider including a service provider description associated with a plumbing service category. In other examples, the need of the person may be identified as attending an appointment and as such, the at least one processor may retrieve a transportation service provider from the service provider database.

Method 200 further includes the at least one processor transmitting 210 a referral message to the caregiver, the referral message includes the need of the person and the service provider retrieved from the service provider database. In some cases, the at least one processor may transmit the referral message to the person.

In other embodiments, the at least one processor may transmit a notification message (e.g., notification message 140) to the caregiver. The notification message may include the need of the person. In other words, the notification message may indicate the need of the person to the caregiver. In some other cases, the notification message may be associated with a confirmation, i.e., indicating that the needs of the person are addressed. For example, the notification message may include a confirmation message indicating that the person has attended an appointment or paid a recurring payment. In some cases the notification message may be transmitted to the person.

In some embodiments, tracking parameters and transaction parameters may be used together. In some cases, the transaction parameter alert criterion may be associated with a tracking parameter alert criterion. For example, the tracking parameter may include scheduling data retrieved from the personal care database and/or from tracking messages obtained from a device associated with the person. The transaction parameter alert criterion may be associated with the scheduling data. For example, the person may be scheduled to attend an appointment with a healthcare service provider on a specific date. As such, the transaction parameter may include a transaction initiated by the person with the healthcare service provider on the specific date. The transaction parameter alert criterion may include an absence of any transaction with the healthcare service provider on the specific date. In other words, the payment transaction initiated with the healthcare service provide may serve as an indication that the person has attended an appointment. In this example, in response to the transaction parameter satisfying the transaction parameter criterion, then the at least one processor may determine the need of the person includes rescheduling the appointment with the healthcare service provider. Further, the at least one service provider may transmit a referral message to the caregiver identifying the service provider (i.e., the healthcare provider) and the need to reschedule.

FIG. 3 illustrates an example configuration of a server computing device 300 that may be used to implement caregiving computing device 110 (shown in FIG. 1). Computing device 300 includes a processor 302 for executing instructions. Instructions may be stored to a memory 304. Processor 302 may include one or more processing units (e.g., in a multi-core configuration) for executing instructions. The instructions may be executed within a variety of different operating systems on data optimizing computing device, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or other suitable programming languages, etc.).

Processor 302 is operatively coupled to a communication interface 306 such that computing device 300 is capable of communication with remote devices. Processor 302 may also be operatively coupled to a storage device 308. For example, storage device 308 is used to implement transaction database 114, service provider database 116, and/or personal care database 115 Storage device 308 is any computer-operated hardware suitable for storing and/or retrieving data. In some embodiments, storage device 308 is integrated in computing device 300. For example, computing device 300 may include one or more hard disk drives as storage device 308. In other embodiments, storage device 308 is external to computing device 300. For example, storage device 308 may include multiple storage units such as hard disks or solid state disks in a redundant array of inexpensive disks (RAID) configuration. Storage device 308 may include a storage area network (SAN) and/or a network attached storage (NAS) system.

In some embodiments, processor 302 is operatively coupled to storage device 308 via a storage interface 310. Storage interface is any component capable of providing processor 302 with access to storage device 608. Storage interface 310 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 302 with access to storage device 308.

Memory 304 may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

Server computing device 300 includes at least one user interface 312 for receiving commands and input from and/or presenting information to a user 314. User interface 312 may, for example, be any component capable of converting and conveying electronic information to and/or from user 314. In some embodiments, user interface 312 includes an output adapter (not shown), such as a video adapter or an audio adapter, which is operatively coupled to processor 302 and operatively coupleable to an output device (also not shown), such as a display device (e.g., a cathode ray tube (CRT), liquid crystal display (LCD), light emitting diode (LED) display, or “electronic ink” display) or an audio output device (e.g., a speaker or headphones). In some embodiments, user interface 312 is configured to include and present a graphical user interface (not shown), such as a web browser or a client application, to user 314. User 312 may display or report via user interface 312, e.g., results generated by one or more of the methods described above. Additionally or alternatively, server computing device 300 includes an input device (also not shown) for receiving input from user 314. User 314 may use input device, without limitation, to initiate or execute one or more methods or processes described above. Input device may include, for example, a keyboard, a pointing device, a mouse, a stylus, a touch sensitive panel (e.g., a touch pad or a touch screen), a gyroscope, an accelerometer, a position detector, a biometric input device, or an audio input device. A single component such as a touch screen may function as both an output device and input device. Additionally or alternatively, server computing device 300 is configured to receive commands to execute one or more of the methods described above from, and/or to transmit results of the methods for display to, a remote device via communication interface 306.

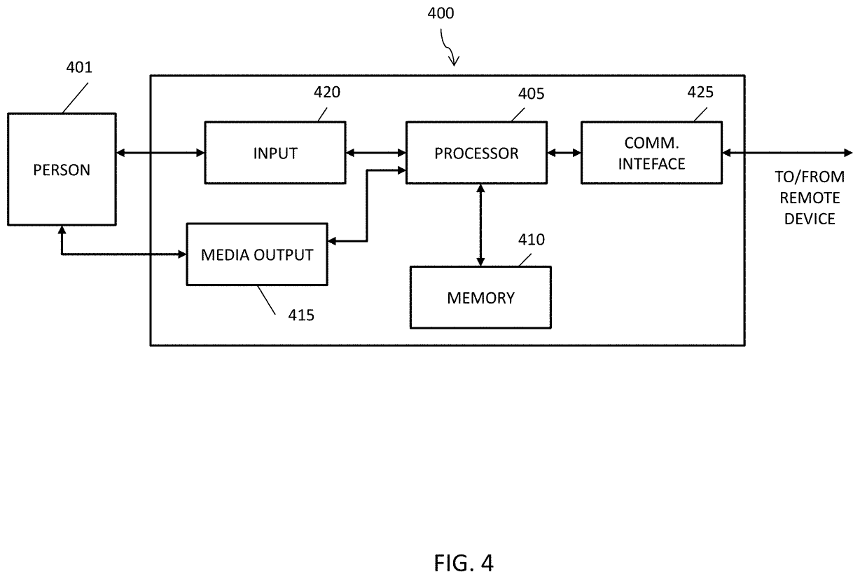

FIG. 4 illustrates an example configuration of a user computing device 400, such as computing device associated with a caregiver (e.g. caregiver 104) and/or a person (e.g. the person 102). The computing device may include laptop, tablet, and/or mobile phone associated with either the person and/or the caregiver. In the example embodiment, user computer device 400, operated by a person 401 (e.g., the person 102 or caregiver 104)). User computer device 400 includes a processor 405 for executing instructions, and a memory 410. In some embodiments, executable instructions are stored in memory 410. Processor 405 may, for example, include one or more processing units (e.g., in a multi-core configuration). Memory 410 may, for example, be any one or more devices allowing information such as executable instructions or transaction data to be stored and retrieved. Memory 410 may further include one or more computer readable media.

In the example embodiment, user computer device 400 further includes at least one media output component 415 for presenting information to customer 401. Media output component 415 may, for example, be any component capable of converting and conveying electronic information to customer 401. In some embodiments, media output component 415 includes an output adapter (not shown), such as a video adapter or an audio adapter, which is operatively coupled to processor 405 and operatively coupleable to an output device (also not shown), such as a display device (e.g., a cathode ray tube (CRT), liquid crystal display (LCD), light emitting diode (LED) display, or “electronic ink” display) or an audio output device (e.g., a speaker or headphones).

In some embodiments, media output component 415 is configured to include and present a graphical user interface such as a web browser or a client application, to customer 401. The graphical user interface may include, for example, an online store interface for viewing or purchasing items, or a wallet application for managing payment information. In some embodiments, user computer device 400 includes an input device 420 for receiving input from customer 401. Customer 401 may use input device 420, without limitation, to select or enter one or more items to purchase or request to purchase, to access credential information, or to access payment information. Input device 420 may include, for example, a keyboard, a pointing device, a mouse, a stylus, a touch sensitive panel (e.g., a touch pad or a touch screen), a gyroscope, an accelerometer, a position detector, a biometric input device, or an audio input device. A single component such as a touch screen may function as both an output device of media output component 415 and input device 420.

In one embodiment, user computer device 400 further includes a communication interface 425, communicatively coupled to a remote device such as server computing system 300 (shown in FIG. 3). Communication interface 425 may include, for example, a wired or wireless network adapter or a wireless data transceiver for use with a mobile telecommunications network.

In the example embodiment, memory 410 stores computer readable instructions for providing a user interface to customer 401 through media output component 415 and, optionally, for receiving and processing input from input device 420. A user interface may include, among other possibilities, a web browser or a client application. Web browsers enable users, such as customer 401, to display and interact with media and other information typically embedded on a web page or a website from server computing device 300. A client application allows person 401 to interact with, for example, server computing device 300. For example, instructions may be stored by a cloud service, and the output of the execution of the instructions sent to the media output component 415.

Processor 405 executes computer-executable instructions for implementing aspects of the disclosure. In some embodiments, the processor 405 is transformed into a special purpose microprocessor by executing computer-executable instructions or by otherwise being programmed.

As will be appreciated based on the foregoing specification, the above-described embodiments of the disclosure may be implemented using computer programming or engineering techniques including computer software, firmware, hardware or any combination or subset thereof, wherein the technical effect or enabling individual chargeback tracking, settlement, and recording. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer system for aiding a caregiver in monitoring the needs of a person, the computer system comprising:
a transaction database storing a plurality of transactions records associated with a plurality of transactions each initiated by one of a plurality of account holders at one of a plurality of merchants, wherein each transaction record includes a merchant identifier associated with the respective merchant and an account identifier associated with the respective account holder, and wherein at least one of the account holders is the person;
a service provider database storing a plurality of service provider records each associated with one of a plurality of service providers, wherein each service provider record includes a service provider identifier, a service provider contact data, and a service provider description associated with the types of goods and/or service provided by the service provider, wherein the service provider description is associated with a need of the person; and
at least one processor in communication with the transaction database and the service provider database, the at least one processor configured to:
monitor a plurality of transaction parameters of the person, including monitoring the transaction database for transaction records including the account identifier associated with the person;
determine if at least one of the plurality of transaction parameters being monitored satisfies a transaction parameter alert criterion,
in response to at least one of the transaction parameters satisfying the transaction parameter alert criterion, determine a first need of the person;
query the service provider database to retrieve a first service provider record having the service provider description associated with the first need of the person; and
transmit a referral message to the caregiver, the referral message including the first need of the person and identifying the service provider associated with the first service provider record.

2. The computer system in accordance with claim 1, wherein the at least one processor is in communication with a personal care database storing a plurality of personal care records, each personal care record including a receiver identifier associated with one of a plurality of care receivers, an item identifier associated with one of a plurality of goods or services required by the respective care receiver, and a schedule data identifying a schedule on which the respective good or service is required, wherein at least one of the plurality of care receivers is the person.

3. The computer system in accordance with claim 1, wherein the at least one processor is further configured to compare the monitored transaction records that include the account identifier associated with the person to at least one personal care record associated with the person to determine whether the person obtained the respective good or service according to the respective schedule data.

4. The computer system in accordance with claim 1, wherein the at least one processor is further configured to determine a scoring parameter for a plurality of merchants in the transaction database for one or more predefined metrics.

5. The computer system in accordance with claim 4, wherein the at least one processor is further configured to identify the service provider from among the merchants associated with the first need of the person based on the scoring parameter, and store the first service provider record associated with the identified service provider to the service provider database.

6. The computer system in accordance with claim 5, wherein the scoring parameter is based on at least one of an average transaction amount per transaction record for transactions occurring with the respective merchant, a number of purchase transactions occurring with the respective merchant over a period of time, and a number of negative transactions occurring with the respective merchant over a period of time.

7. The computer system in accordance with claim 1, wherein the at least one processor is further configured to receive a plurality of tracking messages from a device associated with the person, the tracking messages including a tracking parameter associated with the person.

8. The computing system in accordance with claim 7, wherein the processor is further configured to:
determine if the tracking parameter satisfies a tracking alert criterion; and
in response to the tracking parameter satisfying the tracking alert criterion, transmit a notification message to the caregiver.

9. The computer system in accordance with claim 7, wherein the tracking parameter includes at least one of a location associated with the person, a biometric parameter associated with the person, a blood sugar level associated with the person, and a heart rate associated with the person.

10. A computer-implemented method for aiding a caregiver in monitoring the needs of a person, the method implemented using a computing device including a processor in communication with a memory device for storing data, said method comprising:
a transaction database storing a plurality of transactions records associated with a plurality of transactions each initiated by one of a plurality of account holders at one of a plurality of merchants, wherein each transaction record includes a merchant identifier associated with the respective merchant and an account identifier associated with the respective account holder, and wherein at least one of the account holders is the person;
a service provider database storing a plurality of service provider records each associated with one of a plurality of service providers, wherein each service provider record includes a service provider identifier, a service provider contact data, and a service provider description associated with the types of goods and/or service provided by the service provider, wherein the service provider description is associated with a need of the person; and
at least one processor in communication with the transaction database and the service provider database, the at least one processor configured to:
monitoring a plurality of transaction parameters of the person, including monitoring the transaction database for transaction records including the account identifier associated with the person;
determining if at least one of the plurality of transaction parameters being monitored satisfies a transaction parameter alert criterion,
in response to at least one of the transaction parameters satisfying the transaction parameter alert criterion, determining a first need of the person;
querying the service provider database to retrieve a first service provider record having the service provider description associated with the first need of the person; and
transmitting a referral message to the caregiver, the referral message including the first need of the person and identifying the service provider associated with the first service provider record.

11. The method of claim 10, wherein the at least one processor is in communication with a personal care database storing a plurality of personal care records, each personal care record including a receiver identifier associated with one of a plurality of care receivers, an item identifier associated with one of a plurality of goods or services required by the respective care receiver, and a schedule data identifying a schedule on which the respective good or service is required, wherein at least one of the plurality of care receivers is the person.

12. The method of claim 10, wherein the at least one processor is further configured to compare the monitored transaction records that include the account identifier associated with the person to at least one personal care record associated with the person to determine whether the person obtained the respective good or service according to the respective schedule data.

13. The method of claim 10, wherein the at least one processor is further configured to determine a scoring parameter for a plurality of merchants in the transaction database for one or more predefined metrics.

14. The method of claim 13, wherein the at least one processor is further configured to identify the service provider from among the merchants associated with the first need of the person based on the scoring parameter, and store the first service provider record associated with the identified service provider to the service provider database.

15. The method of claim 14, wherein the scoring parameter is based on at least one of an average transaction amount per transaction record for transactions occurring with the respective merchant, a number of purchase transactions occurring with the respective merchant over a period of time, and a number of negative transactions occurring with the respective merchant over a period of time.

16. The method of claim 10, wherein the at least one processor is further configured to receive a plurality of tracking messages from a device associated with the person, the tracking messages including a tracking parameter associated with the person.

17. The method of claim 16, wherein the processor is further configured to:
determine if the tracking parameter satisfies a tracking alert criterion; and
in response to the tracking parameter satisfying the tracking alert criterion, transmit a notification message to the caregiver.

18. The method of claim 16, wherein the tracking parameter includes at least one of a location associated with the person, a biometric parameter associated with the person, a blood sugar level associated with the person, and a heart rate associated with the person.

19. A non-transitory computer-readable storage medium that includes computer-executable instructions for aiding a caregiver in monitoring the needs of a person, wherein when executed by a data optimization computing device computing device comprising:
a transaction database storing a plurality of transactions records associated with a plurality of transactions each initiated by one of a plurality of account holders at one of a plurality of merchants, wherein each transaction record includes a merchant identifier associated with the respective merchant and an account identifier associated with the respective account holder, and wherein at least one of the account holders is the person;
a service provider database storing a plurality of service provider records each associated with one of a plurality of service providers, wherein each service provider record includes a service provider identifier, a service provider contact data, and a service provider description associated with the types of goods and/or service provided by the service provider, wherein the service provider description is associated with a need of the person; and
at least one processor in communication with the transaction database and the service provider database, the at least one processor configured to:
monitor a plurality of transaction parameters of the person, including monitoring the transaction database for transaction records including the account identifier associated with the person;
determine if at least one of the plurality of transaction parameters being monitored satisfies a transaction parameter alert criterion,
in response to at least one of the transaction parameters satisfying the transaction parameter alert criterion, determine a first need of the person;
query the service provider database to retrieve a first service provider record having the service provider description associated with the first need of the person; and
transmit a referral message to the caregiver, the referral message including the first need of the person and identifying the service provider associated with the first service provider record.

20. The non-transitory computer-readable storage medium of claim 19, wherein the at least one processor is in communication with a personal care database storing a plurality of personal care records, each personal care record including a receiver identifier associated with one of a plurality of care receivers, an item identifier associated with one of a plurality of goods or services required by the respective care receiver, and a schedule data identifying a schedule on which the respective good or service is required, wherein at least one of the plurality of care receivers is the person.
